(19) United States
(12) Patent Application Publication
(10) Pub. No.: US 2006/0013711 A1
(43) Pub. Date: Jan. 19, 2006

(54) Hermetic compresssor

(76) Inventor: Takashi Kakiuchi, Kanagawa (JP)

Correspondence Address: MCDERMOTT WILL & EMERY LLP, 600 13TH STREET, N.W., WASHINGTON, DC, 20005-3096, US

(21) Appl. No.: 10/531,451

(22) PCT Filed: Jun. 17, 2004
(86) PCT No.: PCT/JP04/08820

§ 371 Date: Apr. 14, 2005

(30) Foreign Application Priority Data
Aug. 26, 2003 (JP) 2003-300872

(51) Int. Cl. F04B 35/04; A61M 1/00

(52) U.S. Cl. 417/415; 417/902; 417/437

(57) ABSTRACT

Outer circumferential shape of a balance weight is composed so that the distance between the outer circumference of the balance weight and the piston is substantially constant in the closely approaching interval of the balance weight and piston.

### TECHNICAL FIELD

The present invention relates to a hermetic compressor used in refrigeration cycle, such as refrigerator, air conditioner and freezer.

### BACKGROUND ART

Recently, hermetic compressors used in refrigerators and freezers for household use are strongly demanded to be smaller in size, lower in noise and lower in vibration. In this background, the refrigerant is being shifted to hydrocarbon refrigerant which is natural refrigerant of low global warming coefficient represented by R600a noted for zero ozone depletion coefficient. Besides, to keep balance with the piston which is a main source of vibration, the method of using a balance weight is effective technology for reducing vibrations.

Hitherto, as this kind of hermetic compressor using balance weight, it is attempted to adjust the imbalanced force of the compressor mechanism by equipping a crankshaft with a balance weight of a nearly arc profile.

Referring now to the drawings, a conventional hermetic compressor disclosed in Japanese Laid-open Patent 2000-213462 is described below.

FIG. 5 is a longitudinal sectional view of the conventional compressor. FIG. 6 is a plan sectional view of the conventional compressor.

In FIG. 5 and FIG. 6, closed container 1 is filled with refrigerant 2. Electric motor element 5 composed of stator 3 having winding 3a and rotor 4, and compression element 6 driven by electric motor element 5 are elastically accommodated in container 1 by means of suspension spring 7. Shaft 10 has main shaft body 11 press-fitting rotor 4 and eccentric shaft body 12 formed eccentrically to main shaft body 11. Above eccentric shaft body 12, balance weight 22 of which outer circumference is a nearly arc profile centered on the axial center of main shaft body 11 is fixed. Cylinder block 16 has nearly cylindrical compression chamber 17. Piston 20 is inserted in compression chamber 17 so as to be freely slidable reciprocally. Piston 20 is coupled to eccentric shaft body 12 by means of connecting means 21.

In the hermetic compressor having such configuration, the operation is described below.

Rotor 4 of electric motor element 5 rotates piston 20. As rotary motion of eccentric shaft body 12 is transferred to piston 20 by way of connecting means 21, piston 20 moves reciprocally in compression chamber 17. As a result, refrigerant gas is sucked and compressed in compression chamber 17 from a cooling system (not shown), and discharged again into the cooling system.

At this time of compression action, as piston 20 makes reciprocal motions, reciprocal inertial force is generated as imbalanced force. This reciprocal inertial force is balanced by installing balance weight 22 so as to be in reverse phase to piston 20. In this configuration, the reciprocal inertial force of piston 20 in horizontal direction is canceled to a certain extent.

In the conventional structure, to lower the overall height of the compressor, when balance weight 22 is disposed on a horizontal extension of piston 20, balance weight 22 and piston 20 come to closest distance at the bottom dead center of piston 20. To avoid such interference, balance weight 22 is designed in a nearly arc profile. Accordingly, balance weight 22 does not have sufficient inertial force. That is, reciprocal inertial force of piston 20 cannot be canceled sufficiently, and vibration of the hermetic compressor is increased.

### DISCLOSURE OF THE INVENTION

The invention is devised in the light of the above problems of the prior art, and it is hence an object thereof to present a hermetic compressor of low vibration having a balance weight with a greater inertial force, in a configuration of disposing a balance weight on a horizontal extension of a piston.

The hermetic compressor of the invention comprises (i) an electric moter element, (ii) a compression element driven by the electric moter element, (iii) a closed container accommodating the electric moter element and compression element, and (iv) a refrigerant contained in the closed container. The compression element comprises (i) a shaft having an eccentric shaft body and a main shaft body, (ii) a cylinder block having a compression chamber, (iii) a piston moving reciprocally in the compression chamber, (iv) connecting means for connecting the piston and eccentric shaft body, and (v) a balance weight formed on the shaft. The piston is positioned on a horizontal extension of the balance weight. The outer circumference of the balance weight is formed in such a shape that the distance between the outer circumference of the balance weight and the piston is substantially constant along the closely approaching interval of the balance weight and piston.

### BRIEF DESCRIPTION OF THE DRAWINGS

FIG. 1 is a longitudinal sectional view of hermetic compressor in preferred embodiment of the invention.

FIG. 2 is a plan sectional view of hermetic compressor in the same preferred embodiment.

FIG. 3 is an essential magnified view of hermetic compressor in the same preferred embodiment.

FIG. 4 is an essential model diagram of hermetic compressor in the same preferred embodiment.

FIG. 5 is a longitudinal sectional view of a conventional compressor.

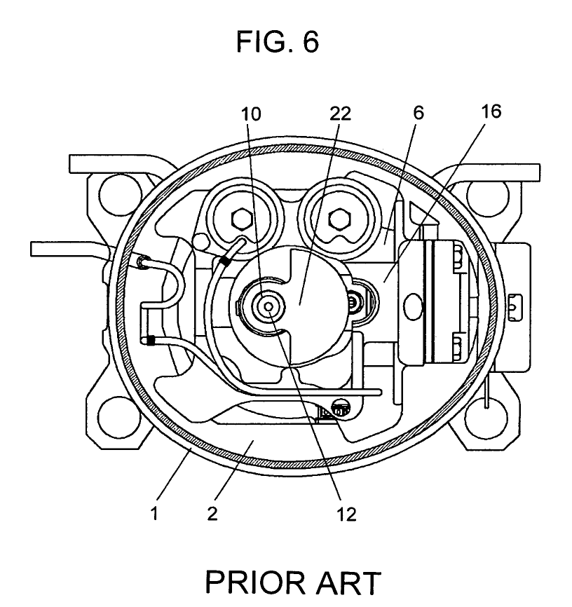

FIG. 6 is a plan sectional view of the conventional compressor.

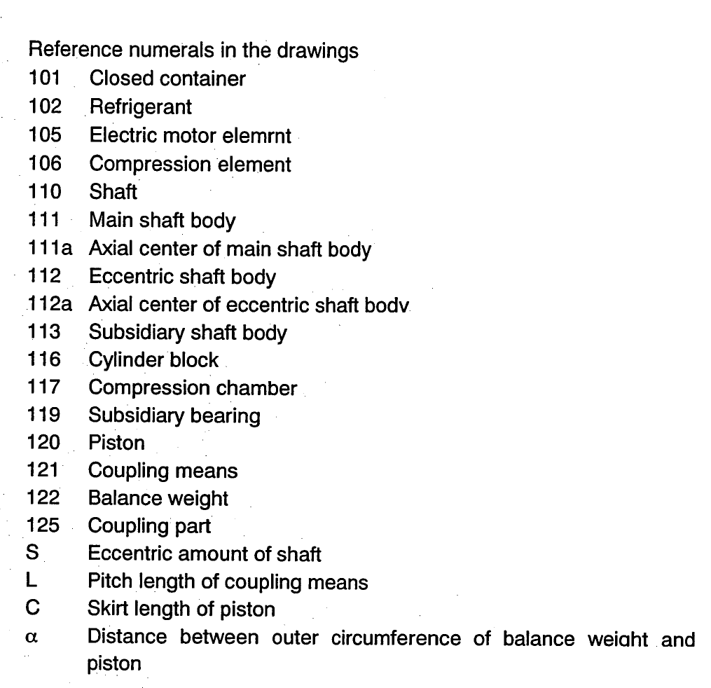

### DETAILED DESCRIPTION OF THE PREFERRED EMBODIMENT

A preferred embodiment of the invention is described specifically below while referring to the accompanying drawings.

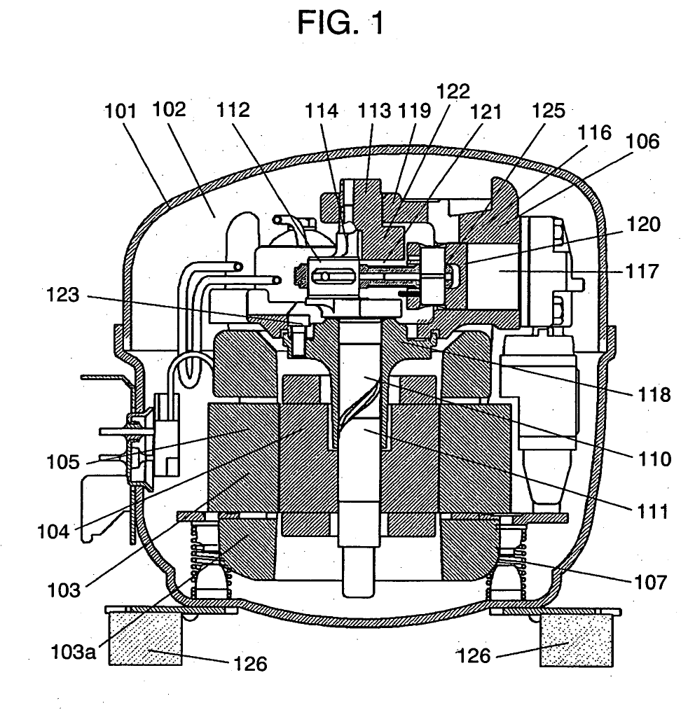

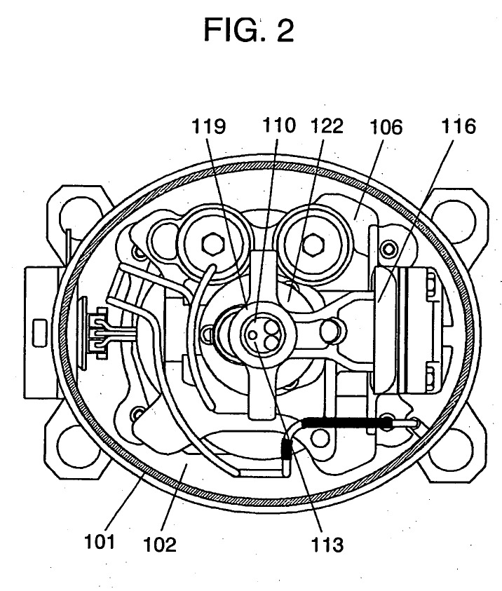

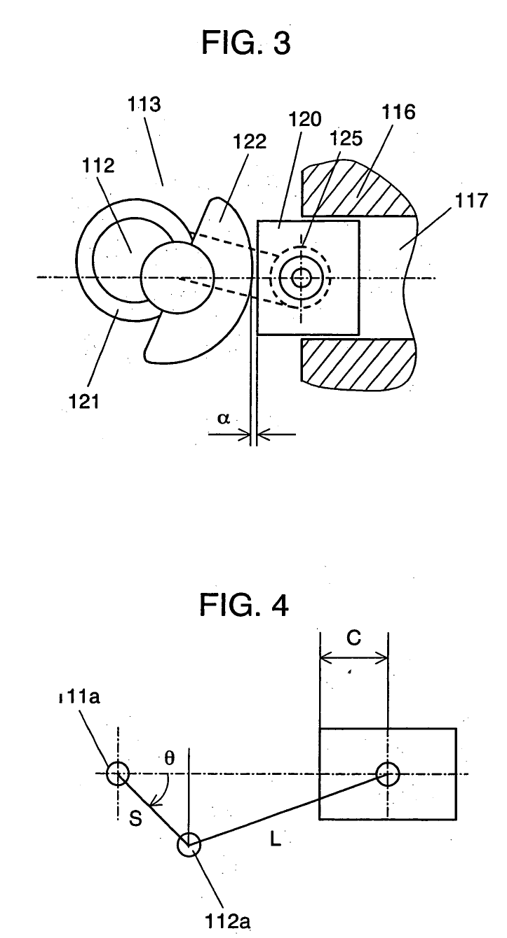

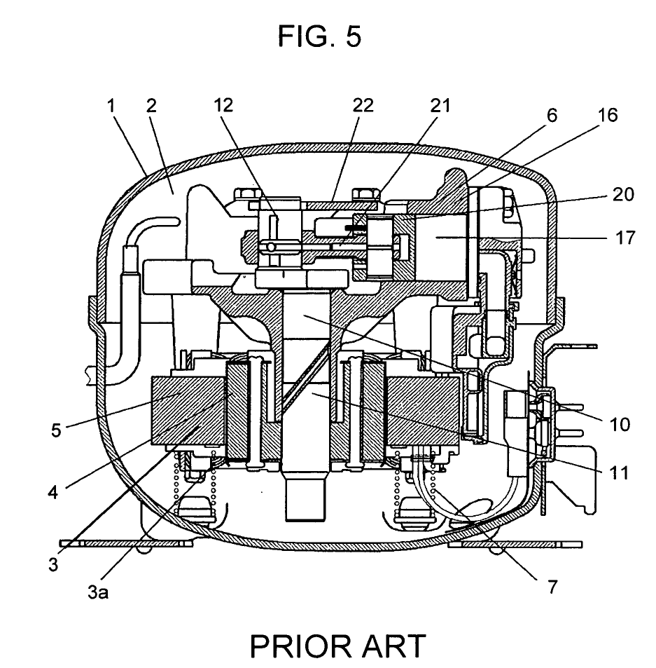

FIG. 1 is a longitudinal sectional view of hermetic compressor in preferred embodiment of the invention. FIG. 2 is a plan sectional view of the same preferred embodiment. FIG. 3 is an essential magnified view of the same preferred embodiment. FIG. 4 is an essential model diagram of the same preferred embodiment.

In FIG. 1 to FIG. 4, closed container 101 is filled with refrigerant 102 composed of isobutane (R600a). Electric moter element 105 composed of stator 103 and rotor 104, and compression element 106 driven by electric moter element 105 are elastically accommodated in closed container 101 by means of suspension spring 107. Electric moter element 105 is driven by inverter at plural operating frequencies including an operating frequency of less than the power source frequency. Herein, a frequency of 30 Hz or less is included in the operating frequency. Closed container 101 is supported by grommet 126.

Shaft 110 has (i) main shaft body 111 press-fitting rotor 104, (ii) eccentric shaft body 112 formed eccentrically to main shaft body 111, (iii) subsidiary shaft body 113 provided coaxially with main shaft body 111, (iv) joint 114 for connecting between eccentric shaft body 112 and subsidiary shaft body 113, and (v) balance weight 122 made of same material as shaft 110 in the lower part of subsidiary shaft body 113. Piston 120 is positioned on a horizontal extension of balance weight 122.

Cylinder block 116 having compression chamber 117 of nearly cylindrical shape has subsidiary bearing 119 for supporting subsidiary shaft body 113 above it. Beneath cylinder block 116, main bearing 118 for supporting main shaft body 111 is fixed by means of screw 123. Piston 120 is slidably inserted in compression chamber 117 of cylinder block 116. Piston 120 is coupled with eccentric shaft body 112 by means of connecting means 121. Supposing axial center Lila of main shaft body 111 to be origin, coordinates (x, y) of outer circumference of balance weight 122 are expressed as (expression-1) and (expression-2).
x=[s·cos (360°−θ)+L·cos {(sin⁻¹(s·sin (360°−θ)/L)}+C−α]·cos (360°−θ) (expression-1)
y=[s·cos (360°−θ)+L·cos {(sin⁻¹(s·sin (360°−θ)/L)}+C−α]·sin (360°−θ) (expression-2)
where s: eccentric amount of shaft 110 (distance between axial center 11a of main shaft body 111 and axial center 112a of eccentric shaft body 112)
L: pitch length of connecting means 121
   C: skirt length of piston 120
   α: distance between outer circumference of balance weight 122 and piston 120
   θ: rotation angle of eccentric shaft body 112

For example, supposing eccentric amount s of shaft 110 to be 10 mm, pitch length L of connecting means 121 to be 37.3 mm, skirt length C of piston 120 to be 9.9 mm, and distance a between outer circumference of balance weight 122 and piston 120 to be 1.5 mm, the coordinates (x, y) of the outer circumference of balance weight 122 are determined specifically as (expression-3) and (expression-4).
x=[10.0×cos (360°−θ)+37.3×cos {(sin⁻¹(10.0×sin (360−θ)/37.3)}+9.9−1.5]×cos (360°−θ) (expression-3)
y=[10.0×cos (360°−θ)+37.3×cos {(sin⁻¹(10.0×sin (360°−θ)/37.3)}+9.9−1.5]×sin (360°−θ) (expression-4)

In this configuration, in the closely approaching interval of balance weight 122 and piston 120, the distance between outer circumference of balance weight 122 and piston 120 may be always kept constant at 1.5 mm. That is, in the structure having balance weight 122 disposed on a horizontal extension of piston 120, in order to utilize effectively the space at the side of shaft 110 of piston 120, by setting distance α at 2.0 mm or less, balance weight 122 having a large mass can be provided. Besides, by defining distance α at 1.5 mm, a sufficient design quality is obtained if considering fluctuations of dimension precision of parts.

The magnitude of inertial force obtained by rotation of balance weight 122 is proportional to the product of the distance from axial center 112a of eccentric shaft body 112 to the center of gravity of balance weight 122 and the mass of balance weight 122. Therefore, according to the preferred embodiment, a greater inertial force can be applied as compared with balance weight 22 of nearly arc profile in the prior art. That is, the reciprocal inertial force of piston 120 can be canceled more effectively than in the prior art, and vibrations can be decreased without sacrificing the downsizing of compressor.

As the refrigerant, hitherto, tetrafluoroethane (R134a) has been generally used, but isobutane (R600a) is used in this preferred embodiment. The density of R600a is small, about 0.6 times that of R134a. Hence, in order to obtain the same refrigerating capacity as R134a, the required cylinder volume is about 1.7 times larger, and the mass of piston 120 is significantly increased. However, the embodiment incorporates balance weight 122 having a large inertial force in a limited space, and the reciprocal inertial force of piston 120 can be sufficiently canceled, and vibrations of the compressor can be decreased.

Besides, to realize high efficiency, the bearing is supported at two sides, and the overall height tends to be higher as compared with the bearing supported at one side. However, in the structure of disposing balance weight 122 on a horizontal extension of piston 120, balance weight 122 having a large inertial force in a limited space can be provided. As a result, the overall height is not so much increased. That is, without sacrificing the downsizing of the compressor, a compressor of high efficiency and low vibration can be presented.

When forming balance weight 122 separately from shaft 110, by employing a process capable of obtaining a dimensional precision close to the die precision such as sinter molding and iron plate presswork, a balance weight of a high dimensional precision can be obtained. As a result, distance a between the outer circumference of balance weight 122 and piston 120 can be shortened. That is, since balance weight 122 having a large inertial force in a limited space can be provided, vibrations of the compressor can be further decreased.

In the case of the bearing supported at both sides, by fixing balance weight 122 formed separately beneath subsidiary shaft body 133 by using bolts or rivets, assembling is easier, and the manufacturing cost of compressor can be lowered.

In the preferred embodiment, cylinder block 116 and main bearing 118 supporting main shaft body 111 are fixed by screws 123, but main bearing 118 may be formed integrally in cylinder block 116. In this case, same effects are obtained.

The smaller end side of connecting means 121 connecting with piston 120 has an annular shape, but a spherical ball joint may be also used. In this case, same effects are obtained.

Nearly same effects as in the invention are obtained by forming notch or dent in part of the outer circumference of balance weight 122.

Electric moter element 105 is driven by inverter at plural operating frequencies including at least a frequency of 30 Hz or less that is an operating frequency of less than power source frequency, by a driving circuit (not shown). As a result, an appropriate refrigerating capacity can be obtained in a refrigerating machine for household use largely fluctuating in load such as refrigerator and freezer.

On the other hand, since electric moter element 105 and compression element 106 are elastically supported by way of suspension spring 107, they have low eigenvalues. By inverter driving at operating frequency of lower than 30 Hz, vibrations of compression element 106 are close to the eigenvalues, and vibrations are increased by resonance. According to the preferred embodiment, since vibrations of compression element 106 can be decreased by applying a large inertial force by balance weight 122, operation at low operating frequency of lower than 30 Hz can be realized.

### INDUSTRIAL APPLICABILITY

As described herein, according to the invention, since the reciprocal inertial force of the piston can be canceled sufficiently by providing a balance weight having a sufficient inertial force in a limited space, on a horizontal extension of the piston, vibrations of hermetic compressor can be decreased. It hence presents a hermetic compressor of low vibration type which can be connected to a refrigeration cycle of refrigerator, air conditioner or freezer.

1. A hermetic compressor comprising:
an electric motor element;
a compression element driven by the electric motor element;
a closed container accommodating the electric motor element and compression element; and
a refrigerant contained in the closed container,
the compression element comprising:
a shaft having an eccentric shaft body and a main shaft body;
a cylinder block having a compression chamber;
a piston moving reciprocally in the compression chamber;
connecting means for connecting the piston and the eccentric shaft body; and
a balance weight formed on the shaft,
wherein the piston is positioned on a horizontal extension of the balance weight; and
wherein the balance weight is formed in such a shape that the distance between the outer circumference of the balance weight and the piston is substantially constant in the closely approaching interval of the balance weight and piston.

2. The hermetic compressor of claim 1,
wherein supposing axial center of the main shaft body to be origin, x-coordinate and y-coordinate of outer circumference of the balance weight are substantially expressed as follows:
x=[s·cos (360°−θ)+L·cos {(sin⁻¹(s·sin (360°−θ)/L)}+C−α]·cos (360°−θ)
y=[s·cos (360°−θ)+L·cos {(sin⁻¹(s·sin (360°−θ)/L)}+C−α]·sin (360°−θ)
where s: distance between axial center of main shaft body and axial center of eccentric shaft body,
L: pitch length of connecting means,
C: skirt length of piston,
α: distance between outer circumference of balance weight and piston, and
θ: rotation angle of eccentric shaft body.

3. The hermetic compressor of claim 1,
wherein the distance between outer circumference of the balance weight and the piston is 2 mm or less.

4. The hermetic compressor of claim 1,
wherein the balance weight is formed by either sinter alloy or press processing of iron plate.

5. The hermetic compressor of claim 1,
wherein the refrigerant is R600a.

6. The hermetic compressor of claim 1, further comprising:
a subsidiary shaft body formed coaxially with the main shaft body; and
a subsidiary bearing for supporting the subsidiary shaft body,
wherein the balance weight is provided at the end of the eccentric shaft body side of the subsidiary shaft body.

7. The hermetic compressor of claim 1,
wherein the electric motor element is driven by inverter at plural operating frequencies including at least an operating frequency of less than the power source frequency.

8. The hermetic compressor of claim 7,
wherein the operating frequency includes at least a frequency of less than 30 Hz.

9. The hermetic compressor of claim 2,
wherein the distance between outer circumference of the balance weight and the piston is 2 mm or less.

10. The hermetic compressor of claim 2,
wherein the balance weight is formed by either sinter alloy or press processing of iron plate.

11. The hermetic compressor of claim 2,
wherein the refrigerant is R600a.

12. The hermetic compressor of claim 2, further comprising:
a subsidiary shaft body formed coaxially with the main shaft body; and
a subsidiary bearing for supporting the subsidiary shaft body,
wherein the balance weight is provided at the end of the eccentric shaft body side of the subsidiary shaft body.

13. The hermetic compressor of claim 2,
wherein the electric motor element is driven by inverter at plural operating frequencies including at least an operating frequency of less than the power source frequency.

14. The hermetic compressor of claim 13,
wherein the operating frequency includes at least a frequency of less than 30 Hz.
